(19) United States
(12) Patent Application Publication
(10) Pub. No.: US 2007/0269830 A1
(43) Pub. Date: Nov. 22, 2007

(54) Process for the Identification of New Medical Targets

(76) Inventors: Giulio Superti-Fuga, Heidelberg (DE); Ulrich Kruse, Dossenheim (DE); Vladimir Rybin, Heidelberg (DE); Gitte Neubauer, Mannheim (DE)

Correspondence Address: CLARK & ELBING LLP, 101 FEDERAL STREET, BOSTON, MA, 02110, US

(21) Appl. No.: 10/555,896

(22) PCT Filed: May 7, 2004
(86) PCT No.: PCT/EP04/04883

§ 371 Date: Dec. 6, 2006

Related U.S. Application Data
(60) Provisional application No. 60/472,663, filed on May 22, 2003.

(30) Foreign Application Priority Data
May 7, 2003 (EP) 03100274.4

(51) Int. Cl. C12Q 1/68; G01N 33/53

(52) U.S. Cl. 435/7.1; 435/6; 436/86

(57) ABSTRACT

The present invention relates to a process for the isolation and identification of pharmaceutically relevant target compounds (TC) from a sample, said sample being derived from a living organism, wherein said target compound(s) bind(s) to a compound of interest (COI) under physiological conditions, said compound of interest (COI) being associated with a given impaired condition or disease. Furthermore, the present invention relates to a process for the identification of a pharmaceutically effective compound useful for preventing and/or treating a given impaired condition or disease, wherein said compound is identified by its binding to a relevant target compound (TC) that has been identified and isolated according to the above mentioned process.

The present invention relates to a process for the isolation and identification of pharmaceutically relevant target compounds (TC) from a sample, wherein said target compound(s) bind(s) to a compound of interest (COI) under physiological conditions, said compound of interest (COI) being associated with a given impaired condition or disease. Furthermore, the present invention relates to a process for the identification of a pharmaceutically effective compound useful for preventing and/or treating a given impaired condition or disease, wherein said compound is identified by its capacity to bind to a relevant target compound (TC) that has been identified and isolated according to the invention.

Most cellular processes are carried out by multiprotein complexes. The identification and analysis of their components provides insight into how the ensemble of expressed proteins (proteome) is organized into functional units. It is the challenge of postgenomic biology to understand how genetic information results in the concerted action of gene products in time and space to generate function. Dissecting the genetic and biochemical circuitry of a cell is a fundamental problem in biology. At the biochemical level, proteins rarely act alone; rather they interact with other proteins to perform particular cellular tasks. Present knowledge regarding the identity of the building elements of specific complexes is limited and is based on selected biochemical approaches and genetic analysis. Previous comprehensive protein-interaction studies are based on ex vivo and in vitro systems, such as, for example, the 2-hybrid systems (see e.g. Uetz et al., Nature 10: 623-7 (2000), reviewed in Uetz et al., Curr. Opin. Chem. Biol. 6: 57-62 (2002), patent application WO 00/60066) and need to be integrated with more physiological approaches.

Seraphin, B. and Rigaut, G. (EP 1 105 508 B1) provide a new approach for detecting and/or purifying biomolecules and/or protein complexes. Their method for purifying biomolecules and/or protein complexes comprises three steps:
(a) providing an expression environment containing one or more heterologous nucleic acids encoding one or more sub-units of a biomolecule complex, the sub-units being fused to at least two different affinity tags, one of which consists of one or more IgG binding domains of Staphylococcus protein A,
(b) maintaining the expression environment under conditions that facilitate expression of the one or more subunits in a native form as fusion proteins with the affinity tags, and under conditions that allow the formation of a complex between the one or more subunits and possibly other components capable of complexing with the one or more subunits,
(c) detecting and/or purifying the one or more subunits by a combination of at least two different affinity purification steps each comprising binding the one or more subunits via one affinity tag to a support material capable of selectively binding one of the affinity tags and separating the one or more subunits from the support material after substances not bound to the support material have been removed.

This method is called TAP purification (Tandem Affinity Purification).

Gavin et al. (Functional organization of the yeast proteome by systematic analysis of protein complexes, Nature, vol. 415, Jan. 10, 2002, p. 141-147) successfully employed this TAP technology for purifying multiprotein complexes on a large scale to systematically analyze protein complexes in Saccharomyces cerevisiae. Specifically, they inserted gene-specific cassettes containing a TAP tag, generated by polymerase chain reaction (PCR), which were inserted by homologous recombination at the 3′ end of the genes. Altogether, they processed 1,739 genes. After growing the yeast cells to mid-log phase, assemblies were purified from total cellular lysate by TAP technology. They combined a first high-affinity purification, mild elution using a site specific protease, and a second affinity purification to obtain protein complexes with high efficiency and specificity. The purified protein assemblies were separated by denaturing gel electrophoresis, individual bands being digested by trypsin, analyzed by matrix-assisted laser desorption/ionization-time-of-flight mass spectrometry (MALDI-TOF MS) or Elektro-spray-mass spectrometry and identified by database search algorithms.

Starting with several distinct tagged proteins as entry points to purify a given complex, core components can be identified and validated, whereas more dynamic, perhaps regulatory components, may be present differentially.

Thus, the TAP technology allowed to assign cellular functions to new, non-annotated gene products, and to understand the context in which proteins operate in yeast. TAP technology allows purification of very large complexes. The success of the TAP/MS approach for the characterization of protein complexes lies in the conditions used for the assembly and retrieval of the complexes. They include maintaining protein concentration, localization and post-translational modifications in a manner that closely approximates normal physiology.

Generally, the early phase of screening methods for identifying medically useful compounds involves some method wherein a screened compound is characterized with respect to its direct binding interaction with a target compound such as a protein, said protein being associated with a given impaired condition or disease. While pharmaceutical companies often have large compound pools in the range of several million individual compounds, there is a growing need for relevant targets for testing these libraries. Today, most often, one specific target compound is known to be associated with one or more specific diseases. However, in most cases it remains unknown if such a given target directly influences a disease or whether it acts indirectly as part of a much larger protein biocomplex, wherein multiple components act together through complex interactions such as cooperative binding, bridging factors, post translational modifications, allosteric structural changes, binding of ions and metabolites to influence the disease process. Because of the complicated and complex interaction of the components involved, it is highly probable that some or even most of the components of a multi-compound biocomplex that is involved in a disease process are potential targets for medical drug screening. The isolation and identification of medically important protein complexes will provide new insight into the molecular basis of many diseases and identify new targets for the therapy and prophylaxis of diseases.

The identification of further binding partners of a given compound of interest that is associated with a given impaired condition or disease (Indication) will either point to (i) a further medical use of the compound of interest when the identified binding partner (by direct and/or indirect binding) has a known medical use, (ii) the identification of new target compounds for drug screening, (iii) potential side effects of the compound of interest when the identified binding partner is known to elicit side effects, or even (iv) the identification of diagnostic agents, when the binding partner is found to be suitable for specific and stable binding of the compound of interest or when the binding partner itself is found indicative of a specific disease or condition.

It is the objective of the present invention to provide a process for isolating and identifying compounds that bind to a compound of interest for the purpose of (i) identifying a further medical use of the compound of interest when the identified binding partner (by direct and/or indirect binding) has a known medical use, (ii) for the identification of new target compounds for drug screening, (iii) for the identification of potential side effects of the compound of interest when the identified binding partner is known to elicit side effects, or even (iv) for the identification of diagnostic agents, when the binding partner is found to be suitable for specific and stable binding of the compound of interest or when the binding partner itself is found indicative of a specific disease or condition.

This problem is solved by providing a process for the isolation and identification of one or more pharmaceutically relevant target compounds (TC) from a sample that directly and/or indirectly bind(s) to a compound of interest (COI), said compound of interest (COI) being associated with a given impaired condition or disease, comprising the following steps:
a) providing said compound of interest (COI), preferably being bound to a suitable solid support material,
b) adding said sample to the compound of interest (COI) from step a), preferably under physiological conditions, resulting in the direct or indirect binding to one or more of the components from said sample (CS) to the compound of interest (COI-CS complex formation),
c) isolating and purifying said complex (COI-CS) from step b) and/or its components,
d) identifying the component(s) of said complex (COI-CS),
e) identifying at least one target compound (TC) of said complex (COI-CS) that is hitherto unknown to directly or indirectly bind to the compound of interest (COI), and optionally
f) further purifying said target compound.

The basic concept underlying the above process is that a compound (COI) that is associated with a given impaired condition or disease is used as a “bait” for its physiological counterpart(s), the target compound(s). Said target compound binds to the bait, and the target compound, preferably the target compound as well as all those compounds with an affinity to said target compound (which are also target compounds) are isolated and purified. It is the affinity of the target compound and/or of the COI to physiologically related compounds that allows for purifying and identifying disease related complexes. For example, according to the present invention one can provide a receptor protein or a small organic drug molecule (COI) that is involved in some impaired condition or disease in a first step, and then add said COI as a bait to a sample, e.g. some mammalian cell lysate, and isolate and purify any complexes under physiological conditions that result from COI-target compound binding. It is critical that if essentially all physiological direct or indirect binding partners of a COI shall be detected that isolation and purification conditions, in particular also the incubation step b) are physiological or at least in the close proximity to the physiological conditions found in the original sample that harbors the target compounds. Under these physiological conditions, not only the COI-target complex is isolated and purified but also all other components that demonstrate affinity to the components of said complex. These co-isolated and purified compounds are potential new compounds (targets) for medical screening assays.

The term “isolation and/or identification” as used herein refers to the isolation and/or identification of a complex comprising at least one compound of interest (COI) being bound to one or more target compounds (TC) and optionally those compounds that demonstrate affinity for said complex. Isolation in this context does not necessarily mean complete purification but merely to a degree of isolation that allows for the identification of at least one of the target compounds associated with said complex.

The term “target compound” (TC) refers to a compound that demonstrates binding affinity to a given compound of interest (COI) directly or indirectly by binding to another target compound that binds directly to a given compound of interest. Target compounds can be any biomolecules such as a protein, peptide, nucleic acid, lipid, small biomolecule or any other molecule present in a living organism. The identified “target compound” may also be used as a “compound of interest” for further studies.

The “target compound” preferentially binds to an active agent of a pharmaceutical composition in vivo.

The term “complex” as used herein refers to a complex of at least one biomolecule (target compound) (TC) with a compound of interest (COI).

The term “compound of interest (COI)” as used herein refers to any type of compound that can be linked to a given impaired condition or disease. Such a COI may be any type of biomolecule, preferably a protein, a peptide, a lipid, a carbohydrate, or a nucleic acid; or any type of a synthetic compound, such as the active agent of a pharmaceutical preparation, preferably a protein, a peptide, a lipid, a nucleic acid, or a synthetic organic drug, more preferably a small molecule organic drug.

Preferably, compounds of interest (COI) are selected from the ROTE LISTE 2003, Arzneimittelverzeichnis für Deutschland, Rote Liste Service GmbH, Frankfurt/Main.

More preferably, said compounds of interest (COI) are associated with diseases selected from cancer; neurodegenerative diseases, preferably Alzheimer's disease or Parkinson's disease; inflammatory diseases, preferably allergies or rheumatoid arthritis; AIDS; metabolic diseases, preferably diabetes mellitus; asthma; arthesiosclerosis; coronary and heart diseases; and infectious diseases.

Most preferred COI's for practicing the present invention are selected from the group consisting of benserazide, sulindac, parthenolide, TNF alpha. These are presently associated with Parkinson's disease (benserazide in combination with Levodopa) and inflammation (the latter three compounds).

Through the identification of new physiological complexes that interact with a given COI, it is now possible to identify components of said complex which can be useful as drug targets and to provide new insight in a disease-related mechanism.

The process of the invention allows to relate known COI to new target compounds, thereby relating these target compounds to the disease or impaired condition that is known to be associated with the COI. These new target compounds are new screening tools for identifying active agents for treating said disease or impaired condition

Moreover, the process of the present invention allows for identifying new uses of given COIs. For example, when a COI forms a complex with one or more target compounds and at least one of these compounds is already known to be associated with a disease or impaired condition that has not yet been associated with the COI, then this is a clear indication that the COI might have potential new drug use.

In a first step, the present invention provides a compound of interest. The compounds of interest may e.g. be selected from any known drug or from any known drug targets or known biologically active product, such as a protein. Preferably, said compound of interest is bound to a solid support material that is suitable to assist the isolation and purification later on during the process of the present invention. In a further preferred embodiment the COI has a reactive moiety that may later be used to attach the COI-bound complex to a solid support or to a further reactive component that assists purification and isolation, e.g. an immunoreactive moiety and an antibody as reactive component leading to immunoprecipitation.

In a further step of the present invention, a sample, preferably being derived from a living organism, is added to the compound of interest under physiological conditions.

“Physiological conditions” for COI-CS (Compound of interest—compounds from said sample/target compounds) formation are essential for practicing the present invention. “Physiological conditions” are inter alia those conditions which were present in the original, unprocessed sample material. Physiological conditions include the physiological protein concentration, pH, salt concentration, buffer capacity and posttranslational modifications of the proteins involved. The term “physiological conditions” does not require conditions identical to those in the original living organism wherefrom the sample is derived but essentially cell-like conditions or conditions close to cellular conditions. A person skilled in the art will of course realize that certain constraints may arise due to the experimental set up which will eventually lead to less cell-like conditions. For example, it will be necessary to destroy cell walls when taking and processing a sample from a living organism to make its components available for COI-binding and complexing. Suitable variations of physiological conditions for practicing the processes of the invention will be apparent to those skilled in the art and are encompassed by the term “physiological conditions” as used herein. Preferably, the sample is processed as a cell lysate or homogenized under mild conditions. In summary, it is to be understood that the term “physiological conditions” relates to conditions close to cell conditions but does not necessarily require that these conditions are identical. (Please, see also Rigaut et al., Nat. Biotechnol. 17, 1030-3 (1999); Puig et al., Methods 24, 218-229 (2001); EP-A-1105508.)

When said sample is added to the COI under physiological conditions, the COI will bind to target compounds in said sample, thus resulting in the direct or indirect binding of one or more of the components from the sample (CS) to the compound of interest (COI-CS complex formation). This complex comprises at least the complex of interest and one potential target compound but may also comprise a multitude of other compounds that demonstrate affinity to the directly bound target compound or the COI. Upon complex formation said complex and/or its associated components are isolated and purified from components which are not associated with the complex. This is done under mild and physiological conditions to leave the complex intact. In a further step, the isolated and purified components of the complex are identified. In a last step, components of said complex that were hitherto unknown to directly or indirectly interact with a compound of interest are identified, and optionally further purified.

Those newly identified target compounds are highly valuable for drug screening for at least two reasons. First, a target compound identified by the process of the invention has strong potential for being used in screening assays for identifying active agents useful for treating the impaired conditions or diseases that are associated with the original compound of interest used for identifying said target compounds. Secondly, if said target compound is identified as a compound that is associated with a disease or an impaired condition that has not hitherto been associated with the compound of interest, then the compound of interest may be of potential drug use for treating the newly associated medical indication. Thus, new medical applications of known active agents can be identified.

In a further aspect the present invention relates to a process wherein said target compounds that are identified according to the process of the present invention are further employed for screening assays for identifying new drugs for treating or preventing impaired conditions or diseases associated with the COI that was used for identifying said target compound.

In a preferred embodiment the present invention relates to a process for the identification of a pharmaceutically effective compound useful for preventing and/or treating a given impaired condition or disease comprising the steps of
(i) selecting one or more pharmaceutically relevant target compounds (TC) from a sample that directly or indirectly bind(s) to a compound of interest (COI), said compound of interest (COI) being associated with a given impaired condition or disease, comprising the following steps:
   a) providing said compound of interest (COI), preferably being bound to a suitable solid support material,
   b) adding said sample to the compound of interest (COI) from step a), preferably under physiological conditions, resulting in the direct or indirect binding of one or more of the components from said sample (CS) to the compound of interest (COI-CS complex formation),
   c) isolating and purifying said complex (COI-CS) from step b) and/or its components,
   d) identifying the component(s) of said complex (COI-CS),
   e) identifying at least one target compound (TC) of said complex (COI-CS) that is hitherto unknown to directly or indirectly bind with the compound of interest (COI);
(ii) employing one or more of the target component(s) (TC) identified in step (i) e) in a screening assay for the identification of pharmaceutically effective compounds.

The term “employing a target compound” for the identification of pharmaceutically effective compounds relates to the use of said target compound in a pharmaceutical screening assay. The skilled person is well aware of how to set up a pharmaceutical screening assay based on the molecular and physiological characteristics of a target compound. Pharmacological validation of potential drug compounds is typically performed by an in vitro binding assay. For example, target compound binding to potential drugs can be measured by competition assays, wherein known binding agents of a given protein compete for protein binding with a potential drug (Competitive binding assay). Surface plasmon resonance can be measured to validate TC-drug binding. Drugs or target compounds can be labeled to identify drug target complexes. In vitro and in vivo activity assays are also useful to validate drugs. For example, if a protein has an enzymatic activity, then the reduction of starting material or the increase of products can be measured or the reduction or increase of cofactors such as NADH/NAD, ATP/ADP, etc. If the potential drug activates or deactivates the target compound's biological function then a cellular functional assay will provide for establishing target compound-drug binding.

In the art, a wide range of techniques are known for establishing assays and screening compound libraries in order to identify potential drugs (Seethala and Fernandes (eds). Handbook of Drug Screening, Marcel Dekker, New York, 2001). Such assays can generally be adapted for rapid screening of large libraries of compounds that were generated by combinatorial chemistry or focused libraries of synthetic or natural compounds.

The most widely used assay techniques are based on radioactivity detection (e.g. scintillation proximity assay) or fluorescent detection technologies (e.g. fluorescence intensity, fluorescence polarization, fluorescence resonance energy transfer or fluorescence correlation spectroscopy).

In the following, preferred pharmaceutical screening assays will be described that can employ one or more of the target component(s) (TC) identified in step (i) e) for the identification of pharmaceutically effective compounds. Each of the preferred assays described below is amendable to high-throughput analysis which facilitates the screening of large numbers of compounds (potential drugs).

In Vitro Binding Assay (Protein-Protein Interaction)

A competitive in vitro binding assay can be used to identify modulators of protein-protein or protein-peptide interactions. These modulators can disrupt the interaction (inhibitors) or stabilize the interaction.

Briefly, a binding assay is performed in which a purified protein (e.g. cyclindependent kinase 2/cyclin E complex) is used to bind a fluorescently labeled peptide. This labeled peptide is contacted with the purified protein in a suitable buffer solution that permits specific binding of the two components to form a protein-peptide complex in the absence of an added chemical compound. Particular buffer conditions can be selected depending of the target protein of interest as long as specific protein-peptide binding occurs in the control reaction. The protein-peptide complex has slow rotational mobility compared to the free peptide which results in a high fluorescence polarization signal. A parallel binding assay is performed in which a chemical compound (test agent) is added to the reaction mixture. If the chemical compound displaces the labeled peptide, the non-bound labeled peptide has a higher rotational mobility than the protein-peptide complex resulting in a lower fluorescence polarization signal. On the other hand, if the chemical compound stabilizes the protein-peptide interaction, an increase in the polarization signal is observed. The amount of labeled peptide bound to the target protein is determined for the reactions in the absence and presence of the chemical compound (test agent). If the amount of bound labeled peptide in the presence of the chemical compound is different than the amount of bound, labeled peptide in the absence of the chemical compound, the compound is a modulator of the interaction between the protein target and the peptide (Pin et al., 1999, Analytical Biochemistry 275, 156-161).

In Vitro Binding Assay (Nuclear Receptor—Hormone Interaction)

A competitive in vitro binding assay can be used to identify modulators of nuclear receptors (e.g., the steroid hormone receptor superfamily). These modulators can stimulate receptor functions (agonists) or block receptor functions (antagonists). A competitive ligand binding assay does not allow to differentiate between the two modes of action.

Briefly, a binding reaction is performed in which a purified human nuclear receptor (e.g., glucocorticoid receptor) is used to bind to a fluorescently labeled hormone ligand (e.g., fluoresceine-dexamethasone). Alternatively, a crude cellular extract containing the receptor can be used in the assay. This labeled ligand is contacted with the purified protein in a suitable buffer solution that permits specific binding of the two components to form a receptor—ligand complex in the absence of an added chemical compound. Particular buffer conditions can be selected depending of the target protein of interest as long as specific receptor—ligand binding occurs in the control reaction.

The protein—ligand complex has slow rotational mobility compared to the free ligand which results in a high fluorescence polarization signal. A parallel binding assay is performed in which a chemical compound (test agent) is added to the reaction mixture. If the chemical compound displaces the labeled ligand, the non-bound labeled ligand has a higher rotational mobility than the receptor—ligand complex resulting in a lower fluorescence polarization signal.

The amount of labeled ligand bound to the target protein is determined for the reactions in the absence and presence of the chemical compound (test agent).

This assay can be used to identify molecules that bind to the receptor but does not allow to distinguish between agonists and antagonists. For further characterization of identified binders a coactivator recruitment assay or a functional cellular assay can be used (see below). (Lin et al., 2002, Anal. Biochem. 300, 15-21; Parker et al., 2000, J. Biomol. Screen. 5, 77-88)

In Vitro Binding Assay (Nuclear Receptor—Coactivator Recruitment)

Ligand-dependent protein—protein interactions between nuclear receptors and nuclear receptor cocativators are important for the biological function of nuclear receptors. An in vitro binding assay based on fluorescence resonance energy transfer can be used to detect and quantify such interactions. This assay format can be used to identify agonists, partial agonist and antagonists.

Briefly, a binding reaction is performed in which a fluorescently labeled nuclear receptor (e.g. estrogen receptor alpha) is used to bind to a fluorescently labeled coactivator or coactivator fragment (e.g. steroid receptor coactivator 1, SRC-1) in the presence of a hormone agonist. Close proximity of the nuclear receptor and coactivator allows transmission of a FRET signal. Compounds disrupting the receptor coactivator complex result in a lower FRET signal (antagonists).

Alternatively, a labeled nuclear receptor and labeled coactivator can be incubated in the absence of a hormone agonist resulting in a low FRET signal. Compounds stimulating the association of receptor and coactivator yield an increased FRET signal (agonists). (Zhou et al., Methods 25, 54-61; Zhou et al., 1998, Mol. Endocrinol. 12, 1594-1604)

In Vitro Enzyme Activity Assay (Protein Kinase)

An in vitro protein tyrosine kinase immunoassay can be used to identify inhibitors of kinase activity.

Briefly, a fluorescein-labeled peptide substrate is incubated with the kinase (e.g. Lck), ATP and an antiphosphotyrosine antibody. As the reaction proceeds, the phosphorylated peptide binds to the antiphosphotyrosine antibody, resulting in an increase in the polarization signal. Compounds that inhibit the kinase result in a low polarization signal.

Alternatively, the assay can be configured in a modified indirect format. A fluorescent phosphopeptide is used as a tracer for complex formation with the antiphospho-tyrosine antibody yielding a high polarization signal. When unlabeled substrate is phosphorylated by the kinase, the product competes with the fluorescent phosphorylated peptide for the antibody. The fluorescent peptide is then released from the antibody into the solution resulting in a loss of polarization signal. Both the direct and indirect assays can be used to identify inhibitors of protein tyrosine kinase activity. (Seethala, 2000, Methods 22, 61-70; Seethala and Menzel, 1997, Anal. Biochem. 253, 210-218; Seethala and Menzel, 1998, Anal. Biochem. 255, 257-262)

This fluorescence polarization assay can be adapted for the use with protein serine/threonine kinases by replacing the antiphophotyrosine antibody with an antiphosphoserine or antiphosphothreonine antibody. (Turek et al., 2001, Anal. Biochem. 299, 45-53, PMID 11726183; Wu et al., 2000, J. Biomol. Screen. 5, 23-30, PMID 10841597).

In Vitro Activity Assay (Protein Phosphatase)

An in vitro protein tyrosine phosphatase immunoassay can be used to identify inhibitors of phosphatase activity.

Briefly, a fluorescein-labeled phosphopeptide substrate is incubated with the phosphatase (e.g., T cell PTP) and an antiphosphotyrosine antibody. As the reaction proceeds, more dephosphorylated peptide is produced which can not bind to the antiphosphotyrosine antibody any more, resulting in a decrease in the polarization signal. For compounds that inhibit the phosphatase the polarization signal remains high.

This fluorescence polarization assay can be adapted for the use with protein serine/threonine phosphatases by replacing the antiphophotyrosine antibody with an antiphosphoserine or antiphosphothreonine antibody. (Parker et al., 2000, J. Biomol. Screen. 5, 77-88)

In Vitro Receptor Binding Assay (GPCR—Ligand Interaction)

An in vitro competitive binding assay can be used to identify modulators (agonists or antagonists) of G protein coupled receptors (GPCRs). Briefly, either intact cells or receptor-containing membrane fragments (e.g., vesicles bearing the CXCR1 receptor) and a fluorescently labeled ligand (e.g., interleukin-8) are incubated such that specific binding occurs. Addition of test compounds can lead to displacement of the labeled ligand resulting in a change of the fluorescence signal as measured by fluorescence polarization or fluorescence correlation spectroscopy). (Klumpp et al., 2001, J. Biomol. Screen. 6, 159-170; Banks et al., 2000, J. Biomol. Screen. 5, 159-168)

Such a binding assay can not differentiate between agonists and antagonists, but identified binders can be further characterized by functional assays that measure production of a second messenger (e.g. cAMP). (Kariv et al., 1999, J. Biomol. Screen. 4, 27-32)

Cellular Functional Assay (Luciferase Reporter Gene System)

A cellular assay can be established to identify modulators of signal transduction. Briefly, a luciferase reporter construct driven by a suitable promoter element (e.g., NFkB reporter) is transfected into a cell line and the luminescence signal is measured in the presence or absence of a cytokines (e.g. interleukin-1 beta). After addition of test agents (chemical compounds) a change of the luminescence signal can be recorded indicating stimulation or inhibition of reporter gene expression. (Davis et al., J. Biomol. Screen. 7, 67-77; Maffia et al., 1999, J. Biomol. Screen. 4, 137-142)

DNA Binding Assay

An exemplary DNA binding assay can be carried out by contacting a complex having DNA binding activity with a radioactive [³²P] end-labeled DNA substrate under appropriate conditions and detecting bound protein. The detection of DNA bound protein can be carried out, e.g., by filtrating the solution through a nitrocellulose filter and determining the radioactivity bound to the filter. This assay is based on the retention of nucleic acid-protein complexes on nitrocellulose whereas free nucleic acid can pass through the filter. (see e.g. Nowock, J. et al., 1982, Methods 30: 607-15)

GTPase Assay

An exemplary GTPase assay can be carried out by loading a complex having GTPase activity with a radioactivity [gamma³²P]-labeled GTP substrate under appropriate conditions and detecting the amount of radioactivity bound to the GTPase protein and the release of free radioactive phosphate. The detection of the remaining GTP substrate bound to the GTPase protein can be carried out, e.g., by filtrating the solution through a nitrocellulose filter and determining the radioactivity bound to the G-protein. (see e.g. Ridley, A. J. et al., 1993, Methods 12: 5151-60)

Protease Assay

An exemplary protease assay can be carried out by contacting a complex having protease activity with a double labeled peptide substrate with fluorine (e.g. EDANS) and quencher chromophores (e.g. DABCYL) under appropriate conditions and detecting the increase of the fluorescence after cleavage.

The substrate contains a fluorescent donor near one end of the peptide and an acceptor group near the other end. The fluorescence of this type of substrate is initially quenched through intramolecular fluorescence resonance energy transfer (FRET) between the donor and acceptor. When the protease cleaves the substrates the products are released from quenching and the fluorescence of the donor becomes apparent. The increase of the fluorescence signal is directly proportional to the amount of substrate hydrolyzed. (see e.g. Taliani, M. et al, 1996, Methods 240: 60-7)

Apoptosis Assay

An exemplary apoptosis assay can be carried out by contacting a complex having apoptosis activity using fluorescent DNA-staining dyes, e.g. propidium iodide, to reveal nuclear morphology substrates under appropriate conditions and detecting the amount of apoptotic cells by confocal or transmission electron microscopy. The detection of apoptotic cells can be carried out by distinguishing viable from apoptotic cells based on morphological alterations typical of adherent cells undergoing apoptosis becoming rounded, condensed, and detached from the dish. (see e.g. Tewari, M. and Dixit, V. M., 1995, J. Biol. Chem., 17 3255-60)

The samples used in the process of the present invention that comprise the potential target compounds are preferably derived from a mammal, preferably from a human, more preferably from a human suffering from said impaired condition or disease.

The term “derive” indicates that the sample is isolated from a mammal and further processed to accommodate the technical constraints of the process of the invention. Samples from healthy mammalian individuals will provide for target compounds at regular expression levels and form complexes with further components of the sample under regular conditions. If samples are taken from humans with an impaired condition or disease, then the compound of interest may be associated with different components and target compounds may be present in different concentrations reflecting the cellular conditions of said mammal.

In a preferred embodiment, the present invention relates to a process, wherein said impaired condition or disease and/or said sample is associated with an impaired condition or disease which is selected from cancer; neurodegenerative diseases, preferably Alzheimer's disease or Parkinson's disease; inflammatory diseases, preferably allergies or rheumatoid arthritis; AIDS; metabolic diseases, preferably diabetes mellitus; asthma; artherioscierosis; coronary and heart diseases; and infectious diseases.

For the formation of the COI-CS complex, it is essential that the complex be formed under physiological conditions. Said physiological conditions include the cellular content of the sample, the protein concentration, the pH, the buffer capacity, osmolarity, temperature of the cells from which the sample is derived. As mentioned before, physiological conditions according to the present invention do not need to be identical to the conditions in complete cells in their natural environment but are merely required to resemble those conditions to an extent that allows for complex formation. Preferably, said physiological conditions for forming a complex of the present invention consider a physiological pH, buffer, and protein content.

Once the complex is formed under physiological conditions care must be exercised not to disrupt said complex when isolating and purifying the complex or its components.

One preferred method of practicing the invention involves affinity labeling of the target compound or the COI prior to step (b) of the process of the present invention. For example, for labeling the TC, cells of the sample, being present, e.g. as whole cells, lysates or extracts, are labeled, e.g. by incubation, with an affinity marker, e.g. a cell permeable affinity marker, e.g. biotinylated parthenolide or biotinylated cell-permeable caspase inhibitor), and then in step (b) said labeled sample is added to the COI for complex formation under physiological conditions. Also, the COI can be labeled by conventional techniques. After optional disruption of the cell (when whole cells were used) the complexes are isolated and purified using solid support material to which the affinity marker has an affinity.

In a preferred embodiment of the present invention, the COI or target compound is bound to a suitable solid support material. This support binding will assist isolation and purification after complex formation.

Preferred solid support materials are Sepharose, such as Sepharose 4B, or agarose or Latex or Cellulose. The matrixes can be coupled by active groups such as NHS, Carbodiimide etc.

In another preferred alternative the processed sample, e.g. lysate, extract, is added to COI's that are bound to solid support. COI's can be coupled to solid support by direct coupling, e.g. amino-, sulfhydryl-, carboxyl-, hydroxyl-, aldehyde-, and ketone groups and by indirect coupling, e.g. via biotin, biotin being covalently attached to COI's and non-covalent binding of biotin to streptavidin which is bound to solid support directly. Linkage to solid support material may involve cleavable and non-cleavable linkers. Isolation and purification of complexes does not necessarily involve the removal of the COI from solid support material. Preferably, the COI-solid support linker is cleavable. More preferably, the linker comprises an enzyme cleavage site. Also preferred is that the linker comprises a site for indirect coupling, more preferably via a hapten or fluorescent dye (e.g. fluorescein covalently bound to drugs such as fluorescein-Taxol, or an antifluoescein antibody bound to protein A beads.) Once the COI-TC complex is formed under physiological conditions while the COI is bound to solid support, the isolation and purification of said complex and its components may proceed.

Preferred binding interfaces for binding the compound of interest to solid support material are linkers with a C-atom backbone. Typically linkers have a backbone of 8, 9 or 10 C-atoms. The linkers contain either, depending on the compound to be coupled, a carboxy- or amino-acive group.

Most preferably, the complexes obtained by a process according to the invention are isolated and purified at least in part by the TAP technology.

A preferred process according to the invention that involves isolation and purification of the COI-CS complex and/or its components in step (c) at least in part by the TAP technology is a process wherein the compound of interest (COI) provided in step (a) is linked to a tandem affinity tag or one or more target compounds (TC) in the sample used in step (b) is linked to a tandem affinity tag.

The term TAP-technology refers to a tandem affinity purification wherein one component of a complex comprising at least two components is provided with two affinity tags. The TAP technology is e.g. disclosed in EP 1105508 B1 and is exemplified by Rigaut et al., Nat. Biotechnol. 17, 1030-3 (1999) and Puig et al. in Methods 24, 218-229 (2001). The tandem affinity purification (TAP) method was used e.g. in: A general procedure for protein complex purification methods 24, 218-229 (2001), Gavin et al. Functional organization of the yeast protein by systematic analysis of protein complexes, Nature, vol. 415, January 2002, 141-147, and Rigaut et al., A generic protein purification method for protein complex characterization and protein exploration, Nature, Biotechnology, vol. 17, October 1999 1030-1032. While the TAP technology has up to now been used mostly for samples, wherein the TAP tag has been added to cell proteins by recombinant methods, the present invention contemplates adding a TAP-tag to the compound of interest or target compounds by any suitable method, such as e.g. synthetic chemical modification or recombinant modification.

According to the invention the TAP-tag may be linked to the COI or the target compounds. For example, TAP-tags may be linked to target compounds in a sample by recombinant techniques such and homologous recombination (see e.g. Gavin et al.) or be linked to COI's by direct or indirect binding (synthetic measures; or recombinant measures, if the COI is a peptide or nucleotide). When a TAP-tag is introduced to target compounds in a sample by recombinant measures, it is preferred to maintain expression of the fusion protein at, or close to, its natural level. Indeed, over-expression of the protein often induces its association with non-natural partners (heat shock proteins, proteazome).

In a more preferred embodiment, the TAP tag consists of 2 IgG binding domains of staphylococcus aureus protein A (Prot A) and a calmodulin binding peptide (CBP), preferably separated by a TEV protease cleavage site. If the COI or the TCs are peptides, such TAP tags can be positioned on the C as well as the N-terminal site of the compound of interest. When using the Prot A module, said module needs to be at the extreme N- or C-terminus of a fusion protein or other compound of interest. Preferably, both affinity tags are selected for highly efficient recovery of proteins present at low concentrations. Prot A binds tightly to an IgG matrix requiring the use of the TEV protease to allude material under native conditions. The eluate of this first affinity purification step is then incubated with calmodulin coated beads in the presence of calcium. After washing, which removes contaminants and the TEV protease remaining after the first affinity selection, the bound material is released under mild conditions with EGTA. Optimized conditions have been developed for the generic use of the TAP strategy. The TAP-tag, however, is very tolerant to buffer conditions and changes to be implemented to optimize recovery of specific complexes.

Once the COI-CS complex according to any process of the invention is formed, either in solution or attached to solid support, the complex and/or its components are isolated and purified from sample components that are not associated with the complex. Appropriate methods, especially for isolating and purifying complexes bound to solid support material are available to the skilled person and comprise e.g. washing, centrifugation, specific affinity purification and elution steps. (e.g. see EP 1 105 508 B1, Rigaut et al., Puig et al., Rigaut et al.)

The isolated and purified material can be analyzed in a number of ways. For a protein complex or component characterization, proteins are preferably concentrated and fractionated, e.g. on a denaturing gel before identification, e.g. mass spectroscopy. Alternatively, Edman degradation or Western blotting may be employed. Because the various purification steps are performed in a gentle native manner, purified complexes or their components may also be tested for their activities or be used in structural analysis.

Preferred methods for identifying complex components are specific antibody binding, perferably immunoprecipitation, Edman degradation or related chemical analysis, Western blot, mass spectroscopy, more preferably matrix-assisted laser desorption/ionization-time-of-light mass spectrometry (MALDI-TOF MS).

The results obtained from the identification techniques are then compared to identify target compounds that have hitherto been unknown to directly or indirectly bind to the compound of interest. This identification can preferably be achieved by comparing the chemical structure and/or physical properties of said component(s) with the information available in sequence databases and/or suitable substance libraries. The person skilled in the art is well aware of how to use modern bioinformatics for identifying known compounds or identifying new compounds.

As mentioned before, the target compounds isolated and identified according to the present invention are useful for screening assays.

Preferably, a screening assay according to the invention comprises
a) contacting one or more target compound(s) (TC) selected in a process according to the invention for isolation and identification of target compounds, with a compound suspected to be pharmaceutically effective, and
b) determining the presence of a chemical and/or physical binding among the compound(s) (TC) and the compound of step a(A).

Compounds suspected to be pharmaceutically effective can be derived from natural sources such as plants, herbs, and animal sources which have been demonstrated to influence mammalian physiology. Typically and preferably, said compounds are selected from a suitable compound library. Such compound libraries are commercially available from e.g. Chemical Diversity Inc., Maybridge, Tripos, Evotec OAI. Most pharmaceutical companies involved in active research have suitable compound libraries in which millions of compounds are stored.

As mentioned before, a process of the present invention is capable of isolating more than just the target compound that actively binds to the compound of interest. Moreover, a process according to the present invention is capable of isolating, purifying and identifying all those components having affinity under physiological conditions with the COI/TC complex.

Therefore, in a preferred embodiment the present invention is also directed to a process for screening medical compounds comprising the contacting of one, some, or all of the components of the identified COI-CS complex.

### EXAMPLE 1

### Identification of the Protein Complex Associated with the Drug Benserazide

Identification of the Interaction between Benserazide and Carbonyl Reductase

Carbonyl reductase was surprisingly identified as a novel drug target in a drug pulldown assay with immobilized benserazide.

Coupling of the Compound of Interest (COI) and Washing of Coupled Beads:

The compound benserazide was immobilized on NHS-activated beads (Affi Gel 10, BioRad) via its NH₂-group. 300 μl of the beads (both with the immobilized. benserazide and control beads) were washed in 10 ml washing buffer A (50 mM Tris, pH 7.5; 0.1 M NaCl, 0.15% lgepal, 1.5 mM MgCl₂, 0.1 mM DTT) for 5-10 min at 4° C. and centrifugation for 5 min at 1000 rpm in a Heraeus Varifuge 3 OR).

Incubation of Beads with Lysate:

Mouse liver cell lysate (60-100 mg total protein) and 125 μl 50× protease inhibitor tablets (Roche, Complete, EDTA free) where added to the beads and the suspension was incubated for 1 h at 4° C. (while rotating). The suspension was washed 1-3 × with 10 ml washing buffer B (50 mM Tris, pH 7.5; 0.1 M NaCl, 0.15% lgepal, 1.5 mM MgCl₂, 0.1 mM DTT, 1× Protease inhibitor tablet (Complete, EDTA free, Roche)) by rotating the suspension for 5-10 min at 4° C. and centrifugation at 10,000 rpm at 4° C. The beads were transferred to a 1 ml MoBiTec column and connected to a 10 ml syringe. 10 ml washing buffer B were added.

Drug Elution:

Elution was performed by adding a 5-10 fold excess of the drug relative to the beads capacity. The drug was dissolved in 500 μl washing buffer B and incubated with beads on a rotating platform for 1 h at 4° C. and subsequently eluted in an eppendorf tube. 300 μl of washing buffer B were added to the beads and eluted immediately in the same eppendorf tube. The beads were washed with 5 ml washing buffer B using a syringe.

Acidic Elution:

500 μl acidic buffer (0.1 M NaOAc, pH 4.0) were added to the beads, the beads were rotated at 4° C. for 10-15 min. After elution in an eppendorf tube the beads were washed with 10 ml H₂O using a syringe.

Boiling of Beads:

300 μl of 2× sample buffer (NuPage LDS sample buffer)+100 mM DTT were added to the beads. After boiling for 10-15 min at 95° C. the suspension was eluted in an eppendorf tube.

The sample was run on a Coomassie gel and the proteins were identified by massspectrometry analysis as described below.

Carbonyl Reductase (CBR1) was Identified as a Binding Partner of Benserazide

Determination of the Inhibitory Effect of Benserazide on Carbonyl Reductase

Determination of Carbonyl-Reductase Activity:

The carbonyl reductase activity was evaluated spectrophotometrically according to the methods of Iwata et al. 1990. Eur. J. Biochem 193, p. 75-81.: Inazu N, Ruepp B., Wirth H., Wermuth B. 1992. BBA, 1116, p. 50-56 and Imamura et al. 1993. Arch. Biochem. Biophys. 300, p. 570-576. The oxidation rate of NADPH was recorded in the presence of the specific substrate menadione at 340 nm at room temperature on a Jenway 6505 UV/VIS Spectrophotometer.

The standard assay mixture consisted of 100 mM sodium phosphate buffer pH 7.0, 0.12 mM NADPH, 0.25 mM menadione. The reaction was started by adding 5-20 μg of E. coli expressed His-tagged human carbonyl reductase (CBR1) or alternatively by adding 10 μl of mouse live lysate extract (total protein concentration of 15 mg/ml). The total volume of the reaction mixture was 1 ml. The change of the absorbance was monitored at 340 nm.

Inhibition of Carbonyl-Reductase Activity with Benserazide:

The inhibition of carbonyl reductase was determined using the assay described in example 1. In addition to menadione as a substrate, the assay mixture was supplemented with 0, 0.5, 1, 2, 3, 6, or 7.5 mM benserazide. The inhibition experiment performed with mouse liver lysate as well as with recombinant CBR1 in protein supplemented probes. The results for mouse liver lysate and recombinant CBR1 are presented in table 1. These results demonstrate that benserazide has a profound inhibitory impact on carbonyl reductase activity.

TABLE 1

initial rate (^{nmol}/ₘᵢₙ)

| benserazide (mM) | carbonyl reductase | mouse liver lysate |
|---|---|---|
| 0 | 24.0 | 16.3 |
| 0.5 | n.d. | 12.1 |
| 1 | n.d. | 8.6 |
| 2 | 13.7 | 1.2 |
| 3 | n.d. | 1.3 |
| 6 | 3.5 | n.d. |
| 7.5 | 0.3 | n.d. |

Identification of Proteins Binding to Carbonyl Reductase

Subsequently, human carbonyl reductase was TAP-tagged at the amino-terminus and expressed in a human neuronal cell line (SK-N-BE2 cells). The protein complex was purified according to TAP-technology procedures (see also O/0009716/EP 1 105 508 B1 and Rigaut, G et.al. (1999), Nature Biotechnology. vol. 17 (10): 1030-1032).

For expression of the TAP-tagged carbonyl reductase, the cell line was infected with a MoMLV-based recombinant virus construct.

For the preparation of the vector, 293 gp cells were grown to 100% confluency. They were split 1:5 on poly-L-lysine plates (1:5 diluted Poly-L-Lysine [0.01% stock solution, Sigma P-4832] in PBS, left on plates for at least 10 min.).

On Day 2 63 μg retroviral vector DNA together with 13 μg of DNA of plasmid encoding an appropriate envelope protein were transfected into 293 gp cells (Somia, N V et al (1999) Proc. Natl. Acad. Sci. USA 96: 12667-12672; Somia, N V et al., (2000) J. Virol. 74: 4420-4424).

On Day 3, the medium was replaced with 15 ml DMEM+10% FBS per 15-cm dish.

On Day 4, the medium containing the viruses (supernatant) was harvested (at 24 h following medium change after transfection). When a second collection was performed, DMEM 10 % FBS was added to the plates and the plates were incubated for another 24 h.

For collecting the supernatant was filtered through a 0.45 micrometer filter (Coming GmbH, cellulose acetate, 431155).

The filter was placed into konical polyallomer centrifuge tubes (Beckman, 358126) that were placed in buckets of a SW 28 rotor (Beckman).

The filtered supernatant was ultracentrifuged at 19400 rpm in the SW 28 rotor for 2 hours at 21° C. The supernatant was discarded. The pellet containing the viruses was resuspended in a small volume (for example 300 μl) of Hank's Balanced Salt Solution [Gibco BRL, 14025-092] by pipetting up and down 100-times using an aerosol-safe tip. These viruses were used for transfection as described below.

Cells that were infected were plated one day before infection into one well of a 6-well plate. 4 hours before infection the old medium on the cells was replaced with fresh medium. Only a minimal volume was added, so that the cells were completely covered (e.g. 700 μl). During infection the cells were actively dividing.

To the concentrated virus, polybrene (hexadimethrine bromide; Sigma, H 9268) was added to achieve a final concentration of 8 μg/ml (this is equivalent to 2.4 μl of the 1 mg/ml polybrene stock per 300 μl of concentrated retrovirus). The virus was incubated in polybrene at room temperature for 1 hour.

For infection, the virus/polybrene mixture was added to the cells and incubated at 37° C. at the appropriate CO₂ concentration for several hours (e.g. over-day or over-night).

Following infection, the medium on the infected cells was replaced with fresh medium. The cells were passaged as usual after they became confluent. The cells contained the retrovirus integrated into their chromosomes and stably expressed the gene of interest.

Purification or Protein Complexes:

For purifying the protein complex associated with carbonyl reductase the following protocols were used.

For the purification of cytoplasmic TAP-tagged proteins 5×10⁸ adherent cells (corresponding to 40 15 cm plates) were used. The cells were harvested and washed 3 times in cold PBS (3 min, 1300 rpm, Heraeus centrifuge). The cells were frozen in liquid nitrogen and stored at −80° C., or the TAP purification was directly continued.

The cells were lysed in 10 ml CZ lysis buffer (50 mM Tris, pH 7.5, 5 % Glycerol, 0.2% IGEPAL, 1.5 mM MgCl₂, 100 mM NaCl, 25 mM NaF, 1 mM Na₃VO₄, 1 mM DTT, containing 1 tablet of protease inhibitor cocktail (Roche) per 25 ml of buffer) by pipetting 2 times up and down, followed by a homogenizing step (10 strokes in a dounce homogenizer with tight pestle). The lysate was incubated for 30 min on ice. After spinning for 10 min at 20000 g the supernatant was subjected to an ultracentrifugation step of 1 h at 100 000 g. The supernatant was frozen in liquid nitrogen and stored at −80° C., or the TAP purification was directly continued.

The lysates were thawn quickly in a 37° C. waterbath. 0.4 ml of unsettled rabbit IgG-Agarose beads (Sigma, washed 3 times in CZ lysis buffer) were added, and incubated for 2 h while rotating at 4° C. Protein complexes bound to the beads were obtained by centrifugation (1 min, 1300 rpm, Heraeus centrifuge). The beads were transferred into 0.8 ml Mobicol M1002 columns (Pierce) and washed with 10 ml CZ lysis buffer (containing 1 tablet of Protease inhibitor cocktail (Roche) per 50 ml of buffer). After an additional washing step with 5 ml TEV cleavage buffer (10 mM Tris, pH 7.5, 100 mM NaCl, 0.1% IGEPAL, 0.5 mM EDTA, 1 mM DTT) the protein-complexes were eluted from the beads by adding 150 μl TEV cleavage buffer, containing 5 μl of TEV-protease (GibcoBRL, Cat. No. 10127-017). For better elution the columns were incubated at 16° C. for 1 h (shaking with 850 rpm). The eluate was applied on fresh Mobicol columns containing 0.2 ml settled calmodulin affinity resin (Stratagene, washed 3 times with CBP wash buffer). 0.2 ml 2 times CBP binding buffer (10 mM Tris, pH 7.5, 100 mM NaCl, 0.1% IGEPAL, 2 mM MgAc, 2 mM imidazole, 4 mM CaCl₂, 1 mM DTT) were added followed by an incubation of 1 h at 4° C. while rotating. Protein-complexes bound to the calmodulin affinity resin were washed with 10 ml CBP wash buffer (10 mM Tris, pH 7.5, 100 mM NaCl, 0.1% IGEPAL, 1 mM MgAc, 1 mM imidazole, 2 mM CaCl₂, 1 mM DTT). They were eluted by the addition of 600 μl CBP elution buffer (10 mM Tris, pH 8.0, 5 mM EGTA) for 5 min at 37° C. (shaking with 850 rpm). The eluates were transferred into a siliconized tube and lyophilized. The calmodulin resin was boiled for 5 min in 50 μl 4× Laemmli sample buffer. The fractions were combined and applied on gradient NuPAGE gels (Invitrogen, 4-12%, 1.5 mm, 10 well).

For the purification of membrane TAP-tagged proteins 5×108 adherent cells (corresponding to 40 15 cm plates) were used. The cells were harvested and washed 3 times in cold PBS (3 min, 1300 rpm, Heraeus centrifuge). The cells were frozen in liquid nitrogen and stored at −80° C., or the TAP purification was directly continued.

The cells were lysed in 10 ml membrane lysis buffer (50 mM Tris, pH 7.5, 7.5% glycerol, 1 mM EDTA, 150 mM NaCl, 25 mM NaF, 1 mM Na₃VO₄, 1 mM DTT, containing 1 tablet of protease inhibitor cocktail (Roche) per 25 ml of buffer) by pipetting 2 times up and down, followed by a homogenizing step (10 strokes in a dounce homogenizer with tight pestle). After spinning for 10 min at 1300 rpm (Heraeus centrifuge) the supernatant was subjected to an ultracentrifugation step of 1 h at 100000 g. The “default” supernatant was frozen in liquid nitrogen and stored at −80° C., or the TAP purification was directly continued. The “membrane” pellet was resuspended in 7.5 ml membrane lysis buffer (+0.8% IGEPAL) by pipetting, followed by resuspension through a gauge needle for 2 times. After incubation for 1 h at 4° C. (while rotating) the lysate was cleared by a centrifugation step of 1 h at 100000 g. The “membrane” supernatant was frozen in liquid nitrogen and stored at −80° C., or the TAP purification was directly continued.

The lysates were thawn quickly in a 37° C. waterbath. 0.4 ml of unsettled rabbit IgG-Agarose beads. (Sigma, washed 3 times in Membrane lysis buffer) were added and incubated for 2 h rotating at 4° C. Protein complexes bound to the beads were obtained by centrifugation (1 min, 1300 rpm, Heraeus centrifuge). The beads were transferred into 0.8 ml Mobicol M1002 columns (Pierce) and the membrane fractions were washed with 10 ml membrane lysis buffer (containing 0.8% IGEPAL and 1 tablet of Protease inhibitor cocktail (Roche) per 50 ml of buffer). The default fractions were treated the same way but the buffer contained only 0.2% IGEPAL. After an additional washing step with 5 ml TEV cleavage buffer (10 mM Tris, pH 7.5, 100 mM NaCl, 0.5 mM EDTA, 1 mM DTT, containing 0.5% IGEPAL for the membrane fraction and 0.1% IGEPAL for the default fraction), the protein-complexes were eluted from the beads by adding 150 μl TEV cleavage buffer, containing 5 μl of TEV-protease (GibcoBRL, Cat. No. 10127-017). For better elution the columns were incubated at 16° C. for 1 h (shaking with 850 rpm). For the membrane fraction 3 additional pi of TEV-protease were added and incubated for another hour. The eluate was applied on fresh Mobicol columns containing 0.2 ml settled calmodulin affinity resin (Stratagene, washed 3 times with CBP wash buffer). 0.2 ml 2 times CBP binding buffer (10 mM Tris, pH 7.5, 100 mM NaCl, 0.1 % IGEPAL, 2 mM MgAc, 2 mM imidazole, 4 mM CaCl₂, 1 mM DTT) was added followed by an incubation of 1 h at 4° C. rotating. Protein-complexes bound to the calmodulin affinity resin were washed with 10 ml of CBP wash buffer (10 mM Tris, pH 7.5, 100 mM NaCl, 0.1 % IGEPAL, 1 mM MgAc, 1 mM imidazole, 2 mM CaCl_{2,} 1 mM DTT). They were eluted by addition of 600 μl CBP elution buffer (10 mM Tris, pH. 8.0, 5 mM EGTA) for 5 min at 37° C. (shaking with 850 rpm). The eluates were transferred into a siliconized tube and lyophilized. The calmodulin resin was boiled for 5 min in 50 μl 4× Laemmli sample buffer. The fractions were combined and applied on gradient NuPAGE gels (Invitrogen, 4-12%, 1.5 mm, 10 well).

The composition of the protein complex was analyzed as described below.

Gel-separated proteins were reduced, alkylated and digested in gel essentially by following the procedure described by Shevchenko et al. (Shevchenko, A., Wilm, M., Vorm, O., Mann, M. Anal. Chem. 1996, 68, 850-858). Briefly, gel-separated proteins were excised from the gel using a clean scalpel, reduced using 10 mM DTT (in 5 mM ammonium bicarbonate, 54° C., 45 min) and subsequently alkylated with 55 mM iodoacetamide (in 5 mM ammonium bicarbonate) at room temperature in the dark (30 min). Reduced and alkylated proteins were digested in gel with porcine trypsine (Promega) at a protease concentration of 12.5 ng/μl in 5 mM ammonium bicarbonate. Digestion was allowed to proceed for 4 hours at 37° C. and the reaction was subsequently stopped using 5 μl 5% formic acid.

Gel plugs were extracted twice with 20 μl 1% TFA and pooled with acidified digest supernatants. Samples were dried in a vaccum centrifuge and resuspended in 13 μl 1% TFA.

Peptide samples were injected into a nano LC system (CapLC, Waters or Ultimate, Dionex) which was directly coupled either to a quadrupole TOF (QTOF2, QTOF Ultima, QTOF Micro, Micromass or QSTAR Pulsar, Sciex) or ion trap (LCQ Deca XP) mass spectrometer. Peptides were separated on the LC system using a gradient of aqueous and organic solvents (see below). Solvent A was 5% acetonitrile in 0.5% formic acid and solvent B was 70% acetonitrile in 0.5% formic acid.

TABLE 2

| Time (min) | % solvent A | % solvent B |
|---|---|---|
| 0 | 95 | 5 |
| 5.33 | 92 | 8 |
| 35 | 50 | 50 |
| 36 | 20 | 80 |
| 40 | 20 | 80 |
| 41 | 95 | 5 |
| 50 | 95 | 5 |

Peptides eluting off the LC system were partially sequenced within the mass spectrometer.

The peptide mass and fragmentation data generated in the LC-MS/MS experiments were used to query fasta formatted protein and nucleotide sequence data-bases maintained and updated regularly at the NCBI (for the NCBInr, dbEST and the human and mouse genomes) and European Bioinformatics Institute (EBI, for the human, mouse, Drosophila and C. elegans proteome databases). Proteins were identified by correlating the measured peptide mass and fragmentation data with the same data computed from the entries in the database using the software tool Mascot (Matrix Science, Perkins, D. N., Pappin, D. J., Creasy, D. M., Cottrell, J. S., Electrophoresis 1999, 20, 3551-67). Search criteria varied depending on which mass spectrometer was used for the analysis.

Proteins identified are:
E1-component of the alpha-ketoglutarate dehydrogenase complex: alpha-ketoglutarate dehydrogenase or oxoglutarate dehydrogenase (OGDH; EC 1.2.4.2)
E2-component of the alpha-ketoglutarate dehydrogenase complex dihydrolipoyl succinyltransferase (OMIM-No. 126063; OMIM:“Online Mendelian Inheritance in Man”, database available at the National Center for Biotechnology Information, www.ncbi.nlm.nih.gov)

E3-component of the alpha-ketoglutarate dehydrogenase complex: dihydrolipoyl dehydrogenase (OMIM-No. 246900)

The α-ketoglutarate dehydrogenase complex is a multienzyme complex consisting of 3 protein subunits, alpha-ketoglutarate dehydrogenase (E1k, or oxoglutarate dehydrogenase; OGDH); dihydrolipoyl succinyltransferase (E2k, or DLST); and dihydrolipoyl dehydrogenase (E3). The complex catalyzes a key reaction in the Krebs tricarboxylic acid cycle.

Alpha-ketoglutarate dehydrogenase (E1k) catalyzes the conversion of alpha-ketogluterate to succinyl coenzyme A, a critical step in the Krebs tricarboxylic acid cycle. Deficiencies in the activity of this enzyme complex have been observed in brain and peripheral cells of patients with Alzheimer's disease.

The DLST gene maps to 14q24.3 and the E3 gene maps to chromosome 7. A second related sequence, possibly a pseudogene, was identified and mapped to chromosome 10, pointed to a possible significance to the finding of a reduction in the activity of this complex in Alzheimer disease brain and cultured skin fibro-blasts from Alzheimer disease patients. (Reference: OMIM 203740).

The association between carbonyl reductase and alpha-ketoglutarate dehydrogenase points to a role of the carbonyl reductase in protecting alpha-ketoglutarate dehydrogenase from inactivation by reactive metabolites.

For example, 4-hydroxy-2-nonenal (HNE) is a highly toxic product of lipid peroxidation. HNE inhibits mitochondrial potent inhibitor of mitochondrial respiration. HNE inhibits alpha-KGDH.

### EXAMPLE 2

### Identification of the Protein Complex Associated with the Drug Parthenolide

Identification of the Interaction between Parthenolide and IKKbeta

Parthenolide is a natural compound that can be isolated from the medicinal herb feverfew (Tanacetum parthenium). It is known from traditional medicine that parthenolide has anti-inflammatory properties. In order to identify the molecular (intracellular) target for this compound a parthenolide affinity reagent was synthesized.

The experimental procedure was carried out as described in Kwok et al. 2001, Chemistry & Biology 8, 759-766)

Biotinylated parthenolide was synthesized by oxidation with selenium dioxide and tert-butylhydroperoxide to produce the allylic alcohol. The next steps were esterification of the allylic alcohol with 12-(Fmoc amino) dodecanoic acid (Mitsunobu conditions), removal of the Fmoc group with tetrabutylammonium fluoride and coupling with biotin using N-[dimethylamino)-1H-1,2,3-triazolo-[4,5-b]pyridino-1-ylmethylene]-N-methylmethanaminimum hexa-fluorophosphate N-oxide/di-isopropylethylamine. The biotinylated parthenolide product was verified by nuclear magnetic resonance (NMR) and electrospray mass spectroscopy.

This affinity reagent was used to isolate proteins that bind to parthenolide from human cervical carcinoma cells (HeLa). Affinity purification utilized steptavidin resin which tightly interacts with biotin (steptavidin-resin pull-down experiment). IKKbeta was identified as a parthenolide binding protein.

Identification of Proteins Binding to IKKbeta

The IKKbeta protein was fused to the TAP-affinity tag and expressed in Hek 293-cells. TAP purification followed by mass spectrometry analysis identified a protein complex that contained the IKKalpha protein.

The identification was carried out essentially as described in Example 1. As a cell line, Hek 293-cells were used.

Screen for Inhibitors of IKKalpha

The IKKalpha protein is a kinase. Kinases are considered a target class that is pharmaceutically attractive. For kinases that play a role in disease pathways enzymatic assays can be designed that allow for the identification of inhibitors. A number of inhibitors against kinases have been developed that have utility in treating diseases (e.g. cancer or inflammation).

In particular, an enzymatic assay for the IKKalpha kinase was described that allowed the identification of small molecule inhibitors (Burke et al., 2003, JBC 278, 1450-1456; PMID: 12403772). In this assay the enzyme-catalysed phosphorylation of a GST-IkappaBalpha substrate was performed by adding purified IKKalpha enzyme and radioactively labeled gamma [³²P]ATP in a suitable buffer. Reaction samples were analyzed by SDS-polyacrylamide gel electrophoresis and the radioactivity incorporated into the substrate protein was quantified by autoradiography.

Alternatively, a 17-amino acid peptide corresponding to amino acids 26-42 of IkappaBalpha can be used as substrate (PMID: 9575145; PMID: 10593898). The samples are analyzed by HPLC analysis (PMID: 9207191 ) and the amount of IKK-catalyzed incorporation of ³²P into the peptide substrate is quantified by liquid scintillation counting.

Alternatively, a non-radioactive kinase assay can be used to identify IKKalpha inhibitors. This assay is fluorescence-based and as a readout the change of fluorescence polarization is measured (PMID: 10803607; PMID: 11020319). This assay can be performed in a homogeneous way, a simple mix-and-read format, where no separation steps are required and therefore can be used for high throughput screening (HTS) of small molecule libraries. The Fluorescence Polarization (FP)-based protein kinase assay uses fluorescein-labeled phosphopeptides bound to an anti-phosphotyrosine antibody (or anti-serine/anti-threonine antibodies for serine/threonine kinases). Phopsphopeptides generated by a kinase compete for this binding. In kinase reactions, polarization decreases with time as reaction products displace the fluorescein-labeled phosphopeptide from the anti-phosphotyrosine (or anti-phosphoserine/threonine) antibodies. For IKKalpha a fluorescein-labeled peptide corresponding to amino acids 26-42 of IkappaBalpha containing phosphoserine at position 32 or 36 is used as tracer molecule. Non-fluorescent non-phosphorylated peptides of the same sequence serve as substrate for the IKKalpha kinase. Once these substrate peptides are phophorylated by the kinase, they displace the fluorescent phosphopeptide tracer from the anti-phosphoserine antibody and the polarization signal decreases.

1.-15. (canceled)

16. A process for the identification of new uses of an active agent comprising the following steps:
a) identifying a binding molecule for the active agent;
b) identifying cellular binding partners of the binding molecule under physiological conditions, and
c) identifying medical indications linked to one or more of the binding partners of step b).

17. The process according to claim 16, wherein said active agent is selected from the group consisting of a synthetic compound.

18. The process according to claim 17, wherein said active agent is a synthetic organic drug.

19. The process according to claim 18, wherein said active agent is a small molecule organic drug.

20. The process according to claim 16, wherein said active agent is an active agent of a pharmaceutical composition.

21. The process according to claim 16, wherein said active agent is selected from the group consisting of benserazide, sulindac or parthenolide.

22. The process according to claim 16, wherein in step a) said active agent is bound to a solid support material.

23. The process according to claim 16, wherein said binding molecule as defined in step a) is a protein that directly or indirectly binds to the active agent.

24. The process according to claim 16, wherein said binding molecule as defined in step a) is part of a sample derived from a mammal.

25. The process according to claim 24, wherein said mammal is suffering from an impaired condition or disease.

26. The process according to claim 24, wherein said mammal is a human.

27. The process according to claim 26, wherein said human is suffering from an impaired condition or disease.

28. The process according to claim 27, wherein said impaired condition or disease is selected from cancer, neurodegenerative diseases, inflammatory diseases, AIDS, metabolic diseases, asthma, arteriosclerosis, coronary and heart diseases, and infectious diseases.

29. The process according to claim 28, wherein said neurodegenerative disease is Alzheimer's disease or Parkinson's disease.

30. The process according to claim 28, wherein said inflammatory disease is an allergy or rheumatoid arthritis.

31. The process according to claim 28, wherein said metabolic disease is diabetes mellitus.

32. The process according to claim 16, wherein step a) is performed under physiological conditions.

33. The process according to claim 32, wherein said physiological conditions in step a) are selected from a physiological cellular protein concentration, a physiological pH value, a physiological buffer capacity, a physiological osmolarity, and a physiological temperature.

34. The process according to claim 16, wherein in step b) said cellular binding partners are identified at least in part by a technique selected from the group consisting of specific antibody binding, peptide-sequencing, Maldi-TOF mass-spectrometry, and electrospray-mass-spectrometry.

35. The process according to claim 16, wherein said cellular binding partners as defined in step b) are proteins that directly or indirectly interact with the binding molecule.

36. The process according to claim 16, wherein said physiological conditions in step b) are selected from a physiological cellular protein concentration, a physiological pH value, a physiological buffer capacity, a physiological osmolarity, and a physiological temperature.

37. The process according to claim 16, wherein in step b) the binding molecule is provided and the binding of the cellular binding partners to the binding molecule is determined.

38. The process according to claim 16, wherein step b) is performed using the TAP purification technology.

39. The process according to claim 16, wherein said medical indications in step c) are selected the group consisting of cancer, neurodegenerative diseases, inflammatory diseases, AIDS, metabolic diseases, asthma, arteriosclerosis, coronary and heart diseases, and infectious diseases.

40. The process according to claim 39, wherein said neurodegenerative disease is Alzheimer's disease or Parkinson's disease.

41. The process according to claim 39, wherein said inflammatory disease is an allergy or rheumatoid arthritis.

42. The process according to claim 39, wherein said metabolic disease is diabetes mellitus.
